SEQUENCE LISTING (3 sequences)

SEQ ID NO: 1 - protein, 97 aa, Homo sapiens
PNHQSGSPTGSSDLLLSGKKQRPHLALRRKRRREMKKINRKVRRMNLDLIKEKTAWQHLQALISEAEEVLKTSQTPQTSLTLFLALLSVLGPPPVTG

SEQ ID NO: 2 - protein, 135 aa, Homo sapiens
MGSSHHHHHHSSGLVPRGSHMASMTGGQQMGRGSEFGMPNHQSGSPTGSSDLLLSGKKQRPHLALRRKRRREMKKINRKVRRMNLDLIKEKTAWQHLQALISEAEEVLKTSQTPQTSLTLFLALLSVLGPPPVTG

SEQ ID NO: 3 - protein, 118 aa, Homo sapiens
GSHMASMTGGQQMGRGSEFGMPNHQSGSPTGSSDLLLSGKKQRPHLALRRKRRREMKKINRKVRRMNLDLIKEKTAWQHLQALISEAEEVLKTSQTPQTSLTLFLALLSVLGPPPVTG

(19) United States
(12) Patent Application Publication
(10) Pub. No.: US 2009/0081220 A1
(43) Pub. Date: Mar. 26, 2009

(54) Method for diagnosing and treatment of breast cancer and pharmaceuticals composition and kit for same

(76) Inventor: Jacob Hochman, Jerusalem (IL)

Correspondence Address: Nath & Associates, 1030 15th. Street, NW, Washington,, DC, 20005, US

(21) Appl. No.: 10/587,835

(22) PCT Filed: Jan. 27, 2005
(86) PCT No.: PCT/IL2005/000105

§ 371 Date: Sep. 20, 2006

Related U.S. Application Data
(60) Provisional application No. 60/539,570, filed on Jan. 29, 2004.

(51) Int. Cl. A61K 39/395; G01N 33/53; A61K 39/44; A61K 39/00

(52) U.S. Cl. 424/138.1; 435/7.23; 424/133.1; 424/178.1; 424/185.1

(57) ABSTRACT

Among other findings, the invention is based on the identification that p14 peptide is expressed in mammary carcinoma cells harboring MMTV. Thus, the invention provides methods for diagnosing breast cancer in subjects comprising determining the levels of expression of the peptide or the levels of anti-p14 antibodies produced in the subject to the peptide. The invention also provides screening methods of individuals with high probability of having breast cancer as well as to therapeutic methods; pharmaceutical compositions and vaccine making use of the anti-p14 antibodies or p14 peptides or produced thereby.

### FIELD OF THE INVENTION

This invention relates to diagnosis, assessment and treatment of cancer, in particular breast cancer.

### LIST OF PRIOR ART

The following is a list of prior art which is considered to be pertinent for describing the state of the art in the field of the invention.
Racevskis, J. J Virol, 64: 4043-4050, 1990;
Yanagawa, S., et al J Virol, 67: 112-118, 1993
Hoch-Marchaim, H., et al. Virology, 242:246-254, 1998;
Hoch-Marchaim, H., Virology, 313:22-32, 2003
Hochman, J., et al. J Cell Biol, 99:1282-1288, 1984
Chan, P. K. Exp Cell Res, 203:174-181, 1992;
Kondo, T., et al. Oncogene, 15:1275-1281, 1997
Etkind, P., et al. Clin Cancer Res, 6:1273-1278, 2000;
Ford, C. E., et al. Clin Cancer Res, 9:1118-1120, 2003;
Wang, Y., et al. Cancer Res, 55:5173-5179, 1995
Liu, B., et al. Cancer Res, 61:1754-1759, 2001

### BACKGROUND OF THE INVENTION

Mouse Mammary Tumor Virus (MMTV) is a type B retrovirus associated primarily with mammary carcinomas in laboratory mice. The most documented association of MMTV with non-mammary tumors is that with T-cell lymphomas [Racevskis, J. J Virol, 64: 4043-4050, 1990; Yanagawa, S., et al J Virol, 67:112-118, 1993]. Previously, it has been demonstrated that the leader peptide of the Env-precursor of MMTV, while present in the cytoplasm, is translocated to and concentrated within the nucleoli of murine T-cell lymphomas that harbor this virus [Hoch-Marchaim, H., et al. Virology, 242:246-254, 1998; Hoch-Marchaim, H., Virology, 313:22-32, 2003]. This peptide, designated p14 based on Western blotting analysis, was purified, sequenced, and found to correspond to the first 97 N-terminal amino acids of the MMTV 73 kDa Env-precursor, terminating just before the beginning of gp52 (see also FIG. 1A). The accurate mass of this protein according to mass spectrometry is 11 kDa. The p14 peptide has been identified and characterized using a monoclonal antibody (M-66) generated against a cell surface epitope of T-25-Adh cells [Hoch-Marchaim, H., et al. 1998, ibid.]. T-25-Adh are non-tumorigenic, immunogenic (substrate adherent) cell variants derived from highly tumorigenic (suspension borne) T-25 cells [Hochman, J., et al. J Cell Biol, 99:1282-1288, 1984]. T-25 cells were derived from the S49 T-cell lymphoma. Monoclonal antibody M-66 also identified an additional peptide (named p21). However, p21 is expressed only in highly tumorigenic S49 cell variants. Other murine lymphoma cells that harbor MMTV and express p14 are devoid of p21 [Hoch-Marchaim, H., et al. 1998, ibid.]. Peptide p21 was suggested to be a C-terminal extension of p14 into the N-terminal region of gp52 (see FIG. 1A) since: (1) Both p14 and p21 are recognized by M-66; and (2) p21, but not p14, is recognized by an anti-MMTV-gp52 antibody [Hoch-Marchaim, H., et al. 1998, ibid.].

Based on colocalization and co-immunoprecipitation studies, p14 binds to the shuttling nucleolar protein B23, implicated in both transport and growth regulation, as well as in other cellular functions [Chan, P. K. Exp Cell Res, 203:174-181, 1992; Kondo, T., et al. Oncogene, 15:1275-1281, 1997]. As in the case of B23, Actinomycin D induces redistribution of p14/21 from the nucleolus to the nucleoplasm. Association with B23 has been previously reported for other auxiliary nucleolar retroviral proteins, such as Rev (HIV) and Rex (HTLV). Indeed, abundant evidence suggests that viruses may target the nucleolus and its components to favor viral transcription, translation and perhaps alter the cell cycle in order to promote virus replication.

Additional interest in MMTV has surfaced in recent years when new evidence suggested an MTV-like retroviral association with human breast cancer [Etkind, P., et al. Clin Cancer Res, 6:1273-1278, 2000; Ford, C. E., et al. Clin Cancer Res, 9:1118-1120, 2003; Wang, Y., et al. Cancer Res, 55:5173-5179, 1995]. It has been established that MMTV-like Env gene sequences that were 95-100% homologous to mouse MMTV sequences were found in about 38% of human breast tumor samples analyzed. These sequences have not been detected in normal breast tissue [Etkind, P., et al. 2000. ibid.]. Furthermore, these sequences have also been detected in T-cell lymphomas of breast cancer patients who were simultaneously diagnosed with both diseases [Etkind, P., et al. 2000. ibid.]. Liu et. al [Liu, B., et al. Cancer Res, 61:1754-1759, 2001] have recently reported the finding of a complete proviral MMTV structure in the genome of human breast cancer tissues. An 86% identity exists between the translated 5′-terminal env sequence (Genbank accession number AF248270) reported by this group with the p14 amino acid sequence.

### GLOSSARY

“Breast cancer” denotes any malignancy developed from cells of the breast. While the disease occurs almost entirely in women, the invention also concerns breast cancer in men. Breast cancer according to the invention includes various types of breast cancer, such as adenocarcinoma, ductal carcinoma in situ (DCIS), lobular carcinoma in situ (LCIS), invasive ductal carcinoma (IDC), invasive lobular carcinoma (ILC), inflammatory breast cancer, medullary carcinoma, mucinous carcinoma, Paget's disease of the nipple, phyllodes tumor and others, as known in the art of oncology. Breast cancer according to the invention is preferably characterized by the presence of p14 peptide in the cell.

It should also be appreciated that the term breast cancer also includes metastases derived from breast cancer which are characterized by the presence of p14 peptide in the metastases.

“Anti-p14 Antibodies” denotes an IgG, IgM, IgD, IgA, and IgG antibody capable of binding to p14 peptide as defined hereinbelow. The definition includes polyclonal antibodies or monoclonal antibodies. This term refers to whole antibodies or “antibody fragments” comprising the antigen-binding domain of the anti-p14 antibodies, e.g. scFv, Fab, F(ab′)2, other antibodies without the Fc portion, diabodies (small bivalent and bispecific antibody fragments), single chain antibodies, other fragments consisting of essentially only the variable, antigen-binding domain of the antibody, etc. it should be understood to those versed in the art that the term anti-p14 antibodies also includes conjugates of the aforementioned anti-p14 antibodies with a targeting moiety or a transfection moiety, facilitating the introduction of the antibody into the cell. One example of a conjugate is an antibody-toxin conjugate. The other member of the conjugate may be bound to anti-p14 antibody directly or via a suitable linker.

“Sample” denotes a solid or fluid bodily sample preferably a biopsy specimen or blood sample. The biopsy specimen includes any sample (tissue or liquid) obtained from a suspicious area in the breast, such as from a breast lump, of a subject suspected of having breast cancer. The biopsy specimen may be a fresh tissue preparation, a cryo-section or paraffin embedded section obtained from the subject's area susceptible of being cancerous. A fluid sample may include a blood sample, including whole blood or blood's serum, or a bodily secretion, e.g. secretion from the mammary glands (e.g. milk), saliva, etc.

“P14 peptide” refers to the MMTV-env leader peptide [Hoch-Marchaim, H., Virology, 313:22-32, 2003]. The term “p14 peptide” according to the invention denotes a p14 peptide comprising the sequence as depicted in SEQ ID NO:1; a fragment of the aforementioned p14 peptide; a homologue of any of the aforementioned p14 peptide; a peptide having at least 90% identity, preferably at least 95% sequence identity, more preferably at least 99% sequence identity, with any of the aforementioned p14 peptide as well as recombinant peptides comprising the aforementioned p14 peptides or fragments. The p14 peptide of the invention may be a naturally occurring, isolated peptide, a semi-synthetic, synthetic or recombinant product.

“Identity” of peptide sequence, as used herein, means that two peptide sequences are identical (i.e., on an amino acid-amino acid basis) over a window of comparison.

The “percentage of sequence identity” is calculated by comparing two optimally aligned sequences over the window of comparison, determining the number of positions at which the identical amino acid residues occurs in both sequences to yield the number of matched positions, dividing the number of matched positions by the total number of positions in the window of comparison (i.e., the window size), and multiplying the result by 100 to yield the percentage of sequence identity.

“Homologous” denotes a peptide sequence which is substantially identical, although not necessarily strictly evolutionarily related, to all or a portion of another peptide sequence derived from a different organism. It is to be understood that a homologous peptide according to the invention is also that being capable of binding to anti-p14 antibodies.

“P14 peptide fragment” denotes a peptide or peptide that has an amino-terminal and/or carboxy-terminal deletion, but where the remaining amino acid sequence is identical to the corresponding positions in the naturally-occurring p14 peptide as depicted in SEQ ID NO:1. It is to be understood that the fragment according to the invention is a functional fragment of the naturally occurring p14 peptide i.e. that is capable of binding to anti-p14 antibodies.

“Recombinant p14 peptide” denotes a p14 peptide which does not exists in nature and which is not associates with all or a portion of the amino acids with which it is associated in the native form or is linked to one or more amino acids other than that to which it is associated in its native form. One example of a recombinant p14 peptide is peptide tagged with His residue(s), as used herein. It should be understood that p14 peptide as defined above is such that it retains the binding characteristics of the native, original peptide, i.e. is capable of binding to anti-p14 antibodies.

At times, the p14 peptide and anti-p14 antibodies will be labeled by joining them, either covalently or non-covalently, with a reporter molecule. A wide variety of reporter molecules are known in the art, such as fluorescentic markers or radioactive markers. The conjugated products thus also form part of the above terms.

“High level of expression” denotes a level significantly (e.g. as determined by statistical determination) higher than a standard. “Standard” as used herein denotes either a single standard value or a plurality of standards with which the level of expression p14 peptide from the tested sample is compared. The standards may be provided, for example, in the form of discrete numeric values or is calorimetric in the form of a chart with different colors or shadings for different levels of expression; or they may be provided in the form of a comparative curve prepared on the basis of such standards. The standards may be prepared by determining the level of expression p14 peptide present in a sample obtained from a plurality of patients positively diagnosed (by other means, for example by a physician, by histological techniques etc.) as having breast cancer at varying levels of severity (being correlated with level of expression of p14). The level of expression for the preparation of the standards may also be determined by various conventional methods such as by pathological techniques. The methods of the invention may be carried out in parallel to a number of standards of healthy subjects and subjects of different cancerous states and the level determined in the assayed sample is then compared to such standards. For example, a peptide content level of between X₁ to X₂ per 1,000,000 cells from a biopsy may be defined as being indicative of grade 1 carcinoma, a higher peptide content of Y₁ to Y₂ per 1,000,000 cells from a biopsy may be defined as being indicative of grade 2 carcinoma, etc. After such standards are prepared, it is possible to compare the level of expression p14 peptide obtained from a specific tested subject to the corresponding value of the standards, and thus obtain an assaying tool.

“Therapeutic effect” denotes any physiological effect, resulting in the treatment or prevention of breast cancer. In the context of the present invention refers to at least one of the following: decrease in tumor size; decrease in rate of tumor growth; stasis of tumor size; decrease in the number of metastasis; decrease in the number of additional metastasis; decrease in invasiveness of the cancer; decrease in the rate of progression of the tumor from one stage to the next, as well as decrease in the angiogenesis induced by the cancer, improved survival, improved life quality of the cancer patient.

Without being limited thereto, “treatment” includes improved survival rate or more rapid recovery from a breast cancer or improvement or elimination of symptoms and other indicators associated with breast cancer or prevention of breast cancer from occurring, preventing the manifestation of such symptoms before they occur, slowing down the progression of breast cancer, slowing down the deterioration of symptoms, slowing down any irreversible damage caused by breast cancer, lessening the severity of breast cancer, improving survival rate or more rapid recovery, as well as preventing breast cancer form occurring, or a combination of two or more of the above.

### SUMMARY OF THE INVENTION

The invention is based on several unexpected findings which paved the way to the development of novel methods and kits for diagnosis and assessment of MMTV-containing lymphomas and mammary carcinomas, as well as for methods of treating the same.

Among others, the invention is based on the finding that:
(a) Polyclonal antibody against recombinant p14, named α-p14, specifically recognizes p14 in both immunofluorescence and Western blot analyses, as well as in paraffin embedded and cryo-sections of different S49 derived tumors;
(b) Two different mammary carcinoma cells harboring MMTV (4T1, and Mm5MT cell lines, ATCC No. CRL-1637 and ATCC Nos. CRL-2539, respectively) as well as a sub-set of human breast carcinoma biopsies express p14 in the nucleoli;
(c) Cellular target proteins for p14 were identified in T-64 cells (derived from and essentially identical to parental T-25 cells, the highly tumorigenic variant of the S49 lymphoma), part of which are of nucleolar/nuclear origin.

Based on the above novel findings it has been realized that p14 peptide may be employed for the diagnosis, assessment (prognosis) and treatment of malignancies, in particular, breast cancer.

A method for diagnosing breast cancer in a subject comprising determining levels of expression of p14 peptide in one or more samples from said subject, a high level of expression signifying a high probability for breast cancer in said subject.

According to one embodiment of the invention, the breast cancer is a subset of cancer characterized by the demonstration (expression) of p14 peptide. Thus, according to this embedment, the methods, kits, and medicaments of the invention are applicable for the treatment of malignancies which are characterized by the expression of p14 peptide.

In accordance with the invention there is also provided a method for screening samples into such which signify that subjects from which they were obtained have a relatively high possibility of having or being susceptible of developing breast cancer and such which signify that subjects from which they were obtained have a relatively lower probability of having or being susceptible of developing breast cancer, the method comprising contacting the samples with anti-p14 antibodies and determining binding of anti-p14 antibodies and p14 peptide in said sample, a high degree of binding signifying a corresponding higher probability of having or being susceptible of developing breast cancer in the subject from which the sample was obtained.

Similarly, the screening method may comprise screening samples by contacting the samples with p14 peptide and determining binding of p14 peptide with anti-p14 antibodies, a high degree of binding signifying a corresponding higher probability of having or being susceptible of developing breast cancer this aspect of the invention there is also provided a kit for performing the above diagnostic method of the invention.

The invention provides a kit for diagnosing breast cancer in a subject comprising anti-p14 antibodies and instructions for use of said anti-p14 antibodies in determining levels of expression of p14 peptide in one or more samples from said subject, a high level of expression signifying a high probability for breast cancer in said subject.

A second kit provided according to the invention for diagnosing breast cancer in a subject comprises p14 peptide and instructions for use of said p14 peptide in determining levels of anti-p14 antibodies in one or more samples from said subject, a high level of anti-p14 antibodies signifying a high probability for breast cancer in said subject.

The invention also provides a therapeutic treatment method comprising administering to a subject in need of anti-cancer treatment an amount of anti-p14 antibodies, the amount being sufficient to achieve an anti cancer effect in said subject.

The present invention provides a method for the treatment of cancer comprising administering to the subject an effective amount of an anti-p14 antibody conjugated to a cytotoxic agent.

The subject may be a patient having breast cancer or a subject which recovered from breast cancer or a subject with high probability of having breast cancer (e.g. based on genetics analysis), the treatment in the latter two cases thus being a prophylactic treatment. The therapeutic effect may be a direct effect on the cancer cells, e.g. inhibition of cell proliferation, or on the immune system of the subject, e.g. production of anti-p14 antibodies which act indirectly on said cancer cells.

Alternatively, there is provided a therapeutic method comprising administering to a subject in need an amount of p14 peptide or an immunogenic portion thereof, the amount being effective to elicit production of anti-p14 antibodies in said subject.

Medicaments comprising anti-p14 antibodies or p14 peptide are also provided.

Accordingly, there is provided a pharmaceutical composition for the treatment of breast cancer comprising as active ingredient an amount of anti-p14 antibodies, the amount being sufficient to achieve a therapeutic effect in said subject.

Further provided is a vaccine comprising as active ingredient an amount of p14 peptide, the amount being sufficient to elicit in a subject production of anti-p14 antibodies.

Finally, the present invention provides the use of anti-p14 antibodies for the preparation of a pharmaceutical composition for treating breast cancer, as well as to the use of p14 peptide for the preparation of a vaccine for eliciting an immune response in a subject, e.g. the production of anti-p14 antibodies.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1A-1B show that p14 is present in mouse mammary carcinomas that harbor MMTV. FIG. 1(A) is a scheme depicting p14 and p21 within the MMTV env precursor peptide; while FIG. 1(B) Exponentially growing cells were lysed in SDS-PAGE sample buffer and run on 15% gels, blotted and probed with α-p14 antibodies as described in Materials and Methods. Rev-2-T-6 and T-64 cells served as controls expressing p14 as well as p14 and p21 respectively. 4T1 (ATCC No: CRL-2539) and Mm5MT (ATCC No: CRL-1637) are mouse mammary carcinoma cell lines.

FIG. 2 shows a comparison of the sequences of p14 and parallel sequence of the provirus found in a human breast carcinoma (AF248270).

FIG. 3A-3F shows p14 peptide in human breast cancer. FIG. 3A-3B show ductal carcinoma; FIGS. 3C-3D show ductal carcinoma in situ; FIGS. 3E-3F are invasive ductal carcinoma. In addition, FIGS. 3A, 3C and 3E are paraffin embedded sections stained with Hematoxylin and Eosin; while FIGS. 3B, 3D and 3F are paraffin embedded sections stained with α-p14, 2^{nd} antibody coupled to peroxidase and counterstained with Hematoxylin. Bar-100 μm.

FIG. 4A-4C show p14 peptide localizes to the nucleolus in human breast cancer. FIGS. 4A-4C show invasive Ductal Carcinoma: FIG. 4A-H&E staining, FIG. 4B-p14 staining, and FIG. 4C-enlarged section from FIG. 4B, respectively. Bars-100 μm and 20 μm for B and C, respectively. Arrows-stained nucleoli.

### DETAILED DESCRIPTION OF THE INVENTION

The novel developments of the present invention are based on the following findings:

(1) p14 Peptide is Present in Mammary Carcinoma Cells.

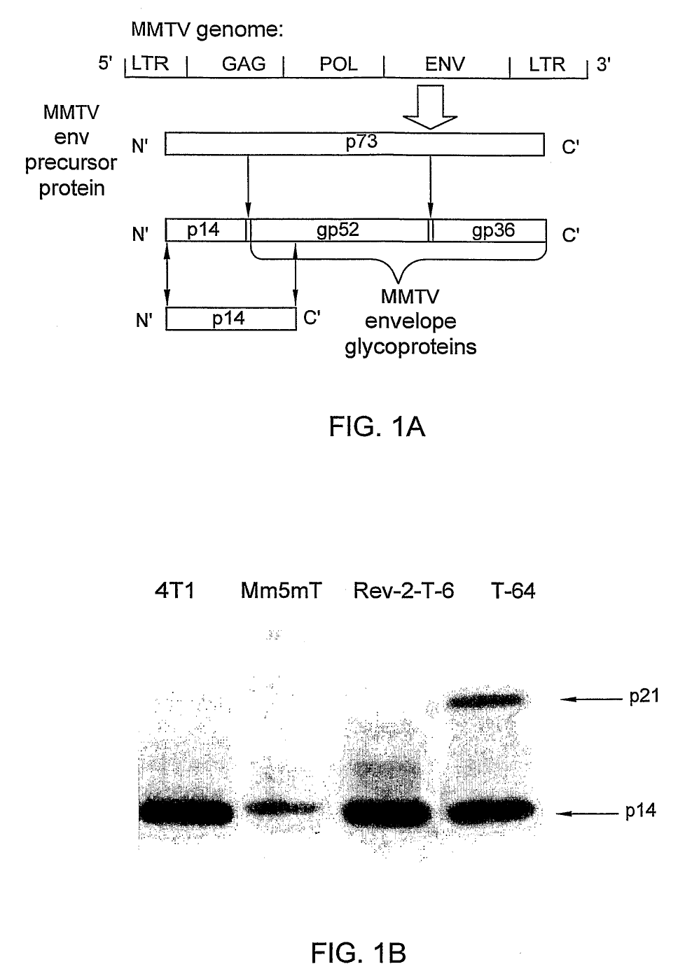

To determine the presence of this peptide in mammary carcinoma cells two murine mammary carcinoma cell lines were analyzed, using the recombinant protein α-p14. Both cell lines (MT1 and Mm5MT, ATCC Nos. CRL-2539 and CRL-1637, respectively) were found to express p14 peptide, but not p21 protein, upon Western blotting analysis (FIG. 1B). Immunofluorescence analysis demonstrated that in both mammary carcinoma cell lines p14 peptide is concentrated in the nucleoli (not shown). In addition, the shuttling nucleolar protein B23 was found to colocalize with p14 in Mm5MT cells (not shown), similar to the co-localization in lymphoma cells [Hoch-Marchaim, H., et al. Virology, 313: 22-32, 2003]. Thus, import of p14 into the nucleoli was determined not to be limited to lymphomas, but also to be a property of MMTV-harboring mammary carcinoma.

(2) p14 is Expressed in Human Breast Cancers.

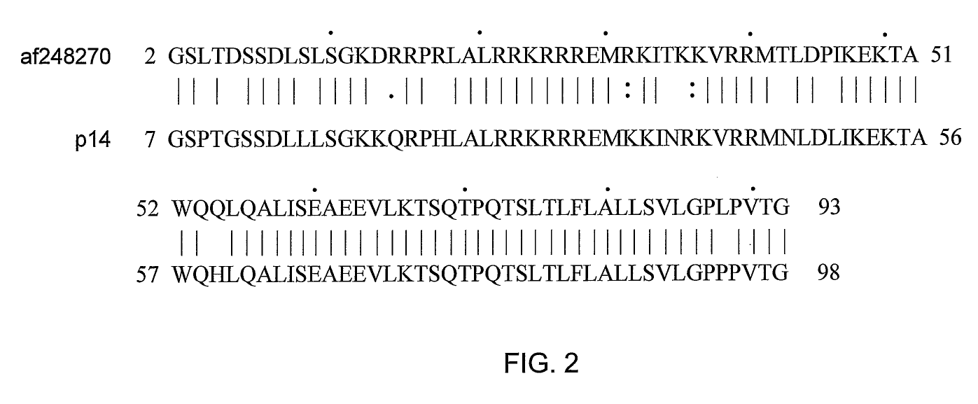

It was found that p14 is also expressed in human breast cancers. FIG. 2 compares the translated 5′-env sequence (Genbank accession number-AF248270) of the proviral MMTV structure found in the genome of human breast cancer tissues [Liu, B., et al. Cancer Res, 61: 1754-1759, 2001], with the p14 amino acid sequence as depicted in SEQ ID NO:1. The two proteins are 86% identical.

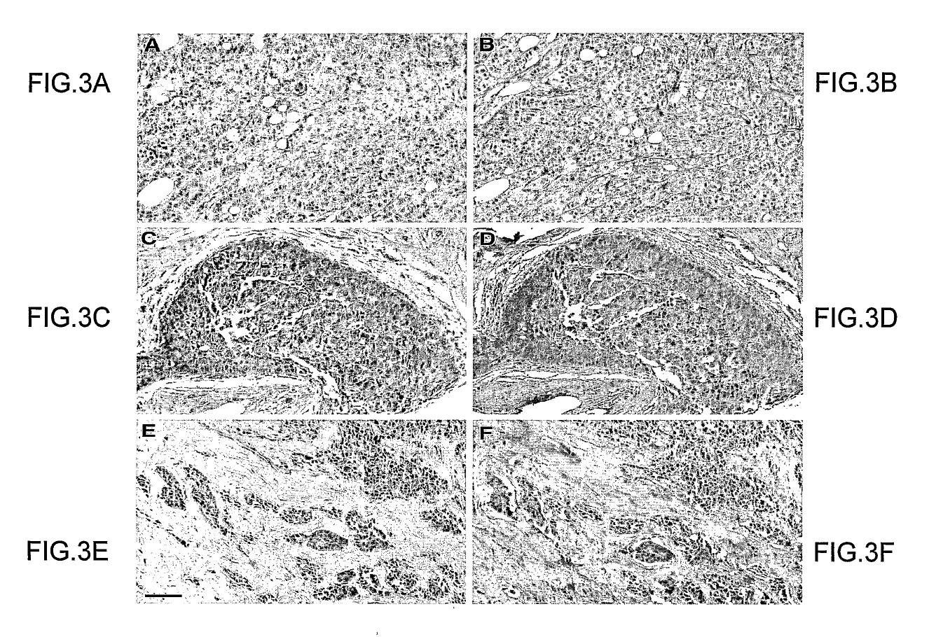

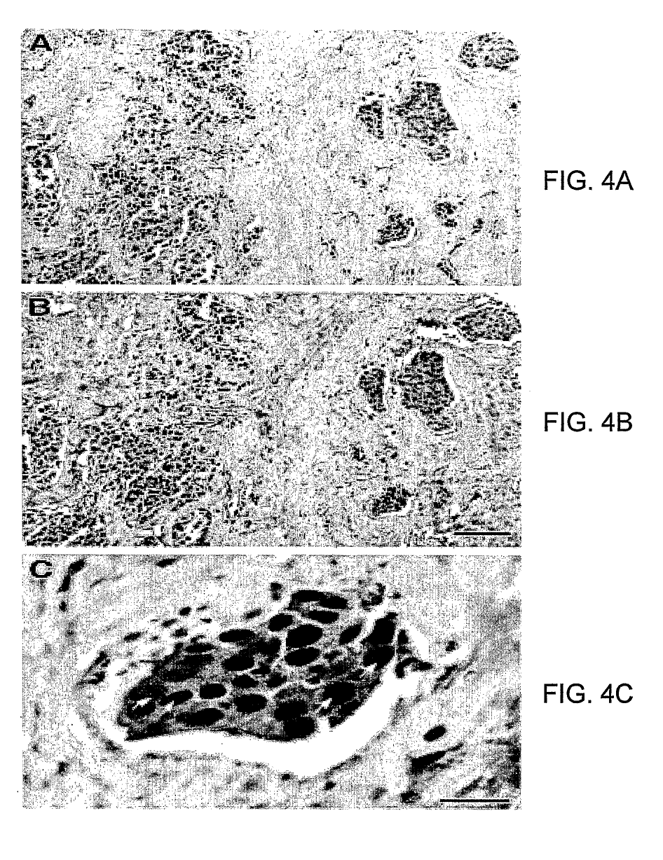

To support this finding, a commercially available tissue array (see Materials and Methods) that contained paraffin embedded sections from 25 human breast cancer biopsies was analyzed. This was carried out using polyclonal α-p14 generated against recombinant p14 (see Materials and Methods). Of the 25 sections probed, two were strongly positive (FIGS. 3D&3F) and two others gave weaker signals (not shown). The rest were negative (for example, FIG. 3B), as were five samples of normal human breast tissue supplied on the same slide. Furthermore, nucleoli of the positive tumor cells are stained for p14 (FIG. 4C). These findings suggest that p14 may be a characteristic feature of a sub-set of human breast cancer, i.e. which express p14. Furthermore, nucleoli of the positive tumor cells are stained for p14 (FIG. 5A-5C). Duplicate slides were stained independently at both the Hebrew University and at the NIH with identical findings.

(3) p14 Peptide has Cellular Targets.

One approach to investigating the function of p14 is to characterize cellular targets that interact with it. T-64 whole cell lysate was adsorbed onto a Co²⁺ column to which recombinant, purified His-tagged p14 (see Materials and Methods) was previously bound. Proteins specifically bound to the affinity column were eluted and sequenced (see Materials and Methods). To gain additional support for the results from the His-tagged p14 Co²⁺ affinity column, another set of experiments was carried out using a different affinity purification procedure. In this experiment the His-tag was removed from recombinant p14 by thrombinization, and the resultant protein was covalently bound to CNBr-activated Sepharose (see Materials and Methods). This column was then used for affinity purification of p14 binding proteins present in T-64 whole cell lysates, as described herein. Proteins identified by both affinity purification procedures are (GenBank accession numbers are in parenthesise): Nucleolar phosphoprotein B23 (Q61937), P31-RACK (AAL77246), Ribosomal protein L5 (NP_{—}058676), La Protein (NP_{—}033304), Nocleosome assembly protein-1 (NP_{—}056596), Glyceraldehyde-3-phosphate dehydrogenase (XP124927) eEFla (XP_{—}203909) and Tubulin α (NP_{—}035784).

Based on the above finding several methods as well as therapeutic methods and compositions have been developed.

According to one aspect, there is provided a method for diagnosing breast cancer in a subject comprising determining levels of expression of p14 peptide in one or more samples from said subject, a high level of expression signifying a high probability for breast cancer in said subject.

The methods of the invention are typically applied to samples obtained from subjects susceptible of having breast cancer, preferably MMTV harboring breast cancer. As appreciated by those versed in the art, the susceptibility of a subject to have breast cancer may be determined by various consideration known in the art such as genetic considerations (e.g. family history of breast cancer in a first degree relative (parent, sibling, child)) or by physical examination (e.g. detection by a specialist of a breast lump, breast enlargement associated with pain, nondescript thickening of the breast etc.).

According to one embodiment, the method comprises assaying for the level of p14 peptide in a sample obtained from the subject, said method comprises contacting said sample with anti-p14 antibodies and determining binding of anti-p14 antibodies to p14 peptide. The sample may be a tissue sample or bodily fluid sample as described hereinbefore.

The tissue sample may be obtained by needle or incisional biopsy or in case the suspicious lesion is small, from an excised lesion suspected of being cancerous. When examining biopsy samples, the sample may be a fresh sample, or a preserved sample (e.g. cryo-section or paraffin embedded).

The method may also be performed using as the sample a blood sample withdrawn from the tested subject. According to this aspect, the blood sample, which may be whole blood or the subject's blood serum, is treated with anti-p14 antibodies as defined above, thereby detecting the presence of p14 peptide in the blood.

The anti-p14 antibodies may be any one of polyclonal or monoclonal antibodies and may also include only one or more binding fragments of the anti-p14 antibody as defined hereinbefore. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse W. D. et al., Science 256:1275-1281, (1989)).

For the production of polyclonal antibodies, various hosts including goats, rabbits, rats, mice, etc. may be immunized by injection with recombinant p14 peptide which retains immunogenic properties. Depending on the host species, various adjuvants may also be used to increase immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful adjuvants. According to the invention polyclonal antibodies may be employed with fresh as well as preserved samples.

A specific polyclonal antibody according to the invention is a polyclonal antibody named herein α-p14 and generated against a recombinant p14 peptide. α-p14 specifically recognized p14 peptide in both immunofluorescence and Western blot analysis, as well as in paraffin embedded and cryo-sections as detailed hereinbelow.

Monoclonal antibodies may be used. According to one embodiment, monoclonal antibodies are preferred when using a fresh biopsy section as the examined sample. Monoclonal antibodies to p14 peptide may be prepared using any technique which provides for the production of antibody molecules by continuous cell lines in culture. These include but are not limited to the hybridoma technique originally described by Koehler and Milstein (Nature 256:495-497, (1975)), the human B-cell hybridoma technique (Kosbor et al., Immunol. Today 4:72, (1983); Cote et al., Proc. Natl. Acad. Sci. 80:2026-2030, (1983)) and the EBV-hybridoma technique (Cole, et al., Mol. Cell. Biol. 62:109-120, (1984)).

After a sufficient time to allow the anti-p14 antibodies to interact with the p14 peptide, if present in the sample, the presence of bound antibodies is determined. The binding of the antibodies to the peptide may be visualized using the relevant fluorescently conjugated secondary antibody (e.g. molecular probes), by the use of a fluorescently labeled affinity purified anti-p14 or its functional fragment, or by the immunohistochemistry analysis. Other determination assays include enzyme-linked immunosorbent assay (ELISA), radioimmunoassay (RIA). A significant binding (i.e. above a predefined threshold or in correlation with predefined reference values) defines the probability of the subject to have mammary carcinoma.

The binding of the p14 peptide, if present in the blood sample, to anti-p14 antibodies, is determined by techniques as described above.

According to a preferred embodiment, when the sample is a blood sample, the anti-p14 antibodies added to the blood sample are polyclonal antibodies, although monoclonal antibodies are also applicable.

According to another embodiment, the method comprises assaying for the level of anti-p14 antibodies in a sample obtained from the subject, the method comprises contacting said sample with p14 peptide and determining binding of p14 peptide to anti-p14 antibodies. One example of a sample applicable according to this embodiment is a blood sample, brought into contact with native (i.e. affinity purified) or recombinant p14 peptide.

P14 peptide may be prepared by techniques known in the art including e.g. using suitable plasmid based techniques (see Material and Methods). A specific recombinant p14 peptide is the N-terminal His-tagged p14 (SEQ ID NO:2) prepared as described hereinbelow.

It should be appreciated that since the presence of antibodies is detected, the above method may be applied for determining active carcinoma as well as whether the tested subject had in the past breast cancer (memory antibody response).

The invention also provides a method for screening samples into such which signify that subjects from which they were obtained have a relatively high possibility of having or being susceptible of developing breast cancer and such which signify that subjects from which they were obtained have a relatively lower probability of having or being susceptible of developing breast cancer, the method comprising contacting the samples with anti-p14 antibodies and determining binding of anti-p14 antibodies and p14 peptide in said sample, a high degree of binding signifying a corresponding higher probability of having or being susceptible of developing breast cancer.

According to this method, the level of p14 peptide in a sample (as defined above) is determined. A variety of protocols for measuring the level of the peptide, using either polyclonal or monoclonal antibodies specific for the p14 peptide are known in the art. Examples include immunohistochemistry, ELISA, RIA, and fluorescent activated cell sorting (FACS).

In a similar screening method, samples are divided by contacting the samples with p14 peptide and determining binding of p14 peptide with anti-p14 antibodies, a high degree of binding signifying a corresponding higher probability of having or being susceptible of developing breast cancer. According to this embodiment the sample is preferably a blood sample. The p14 peptide is preferably a recombinant or native peptide.

The invention also provides a kit for diagnosing breast cancer in a subject comprising either anti-p14 antibodies or p14 peptide and instructions for use of said anti-p14 antibodies or p14 peptide in determining levels of expression of p14 peptide or level of anti-p14 antibodies, respectively, in one or more samples from said subject, a high level of expression signifying a high probability for breast cancer in said subject.

Yet further, the invention concerns therapeutic methods utilizing the anti-p14 antibodies, as an active ingredient. As well appreciated by those versed in the art, in addition to their diagnostic use the antibodies may have a therapeutic utility on the viability of the cancer cells via its effect on the demonstration of p14 peptide in said cells.

The antibody may be conjugated to a cytotoxic agent. Cytotoxic agents are broadly defined to include both toxins and apoptosis-inducing agents. Additionally, for purposes of the invention, cytotoxic gene products include drug metabolizing enzymes which convert a pro-drug into a cytotoxic product. Examples of cytotoxic agents may be used in methods of the invention comprise diphtheria toxin, Pseudomonas toxin, ricin, cholera toxin, PE40 and tumor suppressor genes such as the retinoblastoma gene and p53. Additionally, sequences encoding apoptotic peptides that induce cell apoptosis may be used. Such apoptotic peptides include the Alzheimer's A beta peptide, the calcitonin gene-related peptide, Bcl2 peptides, caspases 3 peptides, BAK, BIK, DF40, as well as other apoptotic peptides known or to be discovered.

Drug metabolizing enzymes which convert a pro-drug into a cytotoxic product include thymidine kinase (from herpes simplex or varicella zoster viruses), cytosine deaminase, nitroreductase, cytochrome p-450 2B1, thymidine phosphorylase, purine nucleoside phosphorylase, alkaline phosphatase, carboxypeptidases A and G2, linamarase, β-lactamase and xanthine oxidase (see Rigg and Sikora, August 1997, Mol. Med. Today, pp. 359-366 for background).

Alternatively, the therapeutic method of the invention comprises administering to a subject in need of anti-cancer treatment an amount of p14 peptide, the amount being sufficient to elicit the production of anti-p14 antibodies which indirectly affect the viability of the cancer cells expressing the peptide.

The anti-p14 antibodies and p14 peptides are administered and dosed in accordance with good medical practice, taking into account the clinical condition of the individual patient, the site and method of administration, scheduling of administration, patient age, sex, body weight and other factors known to medical practitioners.

According to one embodiment, the antibodies employed are preferably humanized monoclonal antibodies. The antibodies are administered typically as a sterile solution by IV injection, although other parenteral routes may be suitable, such as intra tumoral injection. Typically, an antibody administered will be in an amount between about 0.1-5 mg/kg body weight of the subject.

According to another embodiment, the antibody is conjugated to a therapeutic substance. One example of such a conjugate includes antibody-transducing moiety conjugate. The conjugate can be prepared by covalently linking (by chemical reaction), optionally through a linker, the transducing moiety to the anti-p14 antibody or by recombinant techniques. The transduction moieties are known in the art for their ability facilitate or enhance the uptake of substances, such as peptides, into the cell. [Kabouridis P S, Trends in Biotechnology 21(11):498-503 2003]. For example, (37-72)Tat fragment of HIV HV1B1 may be used [Stein S. et al. FEBS LETTERS 456:383-86, 1991], known to facilitate entry of toxins into cells [Mann D A and Frankel A D. Embo J. 10:1733-39, 1991]. Once introduced into the cell, the antibody may affect the activity of p14 peptide within the cell and preferably within the nucleoli.

The treated subject may be a patient diagnosed for having breast cancer, a subject recovered from breast cancer or a subject that has high probability of having mammary carcinoma (e.g. based on genetics analysis), the treatment in the latter two cases thus being a prophylactic treatment.

Thus, in accordance with this aspect, there is also provided a pharmaceutical composition comprising anti-p14 antibodies as an active ingredient, and a physiologically acceptable carrier. The composition according to invention is preferably utilized for treating human breast cancer, more specifically, a sub-set of human breast cancer expressing p14 peptide.

An alternative composition according to the invention is a vaccine (for modulating e.g. activating, stimulating the immune system) comprises an amount of p1⁴ peptide or an immunogenic fragment thereof (i.e. a fragment capable of eliciting an immune response to p14 peptide), the amount being effective to elicit or modulate the immune response to the peptide.

### DESCRIPTION OF SPECIFIC EXAMPLES

### Materials and Methods

### Cells

S49-derived cell lines were grown in Dulbecco's Modified Eagle Medium (DMEM), 10% horse serum, 50 units/ml penicillin and 0.05 mg/ml streptomycin (Biological Industries, Beit-Haemek, Israel) at 37° C. in a humidified atmosphere containing 5% CO₂. Mm5MT (ATCC #CRL-1637) and 4T1 (ATCC#CRL-2539) murine mammary carcinoma cell lines (American Type Culture Collection, Rockville, Md.) were grown in the same medium, but with 10% fetal calf serum.

### N-Terminal His-Tagged p14

The previously isolated cDNA clone 66b [Hoch-Marchaim, H., 1998. ibid.] was used as a template for PCR and the PCR fragment corresponding to the p14 sequence was ligated into the plasmid pET-28a(+) from Novagen (Madison, Wis.). The resulting His-tag p14 plasmid was confirmed by sequencing and transformed into E. coli BL21. The expressed His-tagged peptide was purified to near homogeneity on a TALON™ (Clontech, Palo Alto, Calif.) cobalt-based affinity column. 20 ml bacteria were lysed by sonication in binding buffer (6M guanidine, 50 mM phosphate buffer pH 7.0, 250 mM NaCl), centrifuged @ 13,000 g for 20 min and added to 1 ml beads. The mixture was incubated for 1 hr @ RT, the beads washed three times in 50 mM phosphate buffer pH 7, 300 mM NaCl, 30 mM imidazole. The peptide was eluted from the beads batchwise in the same buffer containing 600 mM imidazole and purity confirmed by SDS-PAGE (SEQ ID NO:2). For studies that did not require the His tag, the tag was removed from the purified peptide by thrombin proteolysis using a thrombin cleavage capture kit (Novagen), to obtain the un-tagged p14 peptide (SEQ ID NO:3).

### p14 Polyclonal Antibody

The above-purified recombinant His-tagged p14 peptide (SEQ ID NO:2) was used to generate polyclonal anti-p14 antibodies in rabbits. 1 mg of the peptide was injected sub-cutaneously into rabbits in Freund's complete adjuvant, followed three and six weeks later by a booster of 1 mg peptide in Freund's incomplete adjuvant. The rabbits were bled three weeks after the second booster. The serum was used with no further fractionation at the specified dilutions.

### Immunofluorescence

Cells were allowed to attach to a polylysine-coated slide (Sigma, St. Louis, Mich.) at 37° C. for 3 hrs in growth medium. The cells were then fixed for 30 min in 4% paraformaldehyde in PBS, permeabilized with 0.1% Triton X-100 in PBS for 3 min. The cells were incubated overnight at 4° C. in the relevant first antibody in PBS containing 5% normal goat serum and 1% BSA (blocking buffer). In double labeling experiments, the two primary antibodies were mixed together. The peptides were visualized using the relevant fluorescently conjugated secondary antibody (Molecular Probes). The fluorescence was visualized in a Zeiss Axiovert 200M microscope outfitted with a Perkin Elmer UltraView confocal scanner (Perkin Elmer, Boston, Mass.). OpenLab software (Improvision) was used to image the data.

### Immunohistochemistry of Human Paraffin-Embedded Breast Cancer Sections

Commercially available paraffin embedded sections from 25 different human breast cancer samples+5 samples of normal human breast tissue, (Chemicon Select Tissue Array: TMA1201-4; Chemicon, Hofhein, Germany), were subjected to immunohistochemical analysis using the polyclonal anti-p14 antibody and the Vectastain ABC kit (Vector, Burlingame, Calif.) according to a standard protocol supplied by the companies. Essentially, the slides were deparaffinized and subjected to epitope retrieval with 10 mM Citrate buffer, pH 6.0. The slides were then treated for 10 minutes with 3% hydrogen peroxide, incubated for 30 min in blocking buffer, and then for 1 hr in anti-p14 polyclonal antibody (1:1000-8000) in blocking buffer. This was followed by staining with the Vectastain ABC kit. The immunohistochemical analysis was also confirmed using a DAKO envision plus kit.

### Western Blots

Cells were lysed by SDS-PAGE sample buffer, separated by 15% SDS-PAGE and transferred to nitrocellulose. p14 and p21 were visualized using the polyclonal anti-p14 described above, HRP-linked donkey anti-rabbit antibody (Jackson Laboratories) and Pierce Super Signal. The amounts of p14 and p21 were quantitated using a FUJI CCD camera and Image Gauge software.

### p14 Binding Proteins

### A. Cell Lysate

Logarithmically growing (1×10⁹) T-64 cells (derived from and essentially identical to parental T-25 cells) were washed in PBS and resuspended in 1.5 ml hypotonic buffer (20 mM Phosphate buffer, pH 7.0, 30 mM NaCl) and sonicated 3×20 seconds. The cell lysate was centrifuged for 20 minutes at 16,000×g, 4° C. and the supernatant (cell lysate) was removed and used in the procedures described below.

### B. Cobalt Bound His-Tagged p14 Affinity Column

1 ml purified His-tagged p14 (250 μg/ml) in binding buffer (6M guanidine, 50 mM phosphate buffer pH 7.0, 250 mM NaCl) was added to 100 μl Talon (Clontech) cobalt-based affinity column beads. The mixture was incubated for 2 hrs. at room temperature with end over end mixing. This was then washed three times with PBS. 1 ml of the cell lysate described above was bound to 20 μl of the affinity column by incubating them end over end for 1 hr at room temperature. The beads were then pelleted and washed with 1.4 ml buffer in each of the following steps:

2× in 30 mM Imidazole, 200 mM NaCl, 40 mM phosphate buffer pH 8.2, 0.05% Tween 20, 1 mM glycine.

2× in 30 mM Imidazole, 200 mM NaCl, 40 mM phosphate buffer pH 7.0.

2× in 30 mM Imidazole, 200 mM NaCl, 40 mM phosphate buffer pH 6.5.

1× in 75 mM NaCl, 20 mM phosphate buffer pH 7.0.

20 μl SDS-PAGE sample buffer was then added to the washed beads, boiled for 1 min and run on a 15% SDS-PAGE gel as described above. The gel was stained with Coomassie brilliant blue and the desired protein bands excised and sent for microsequencing at the Smoler Protein Center, the Technion, Haifa, Israel as previously described (4).

### C. Covalently Bound p14 Affinity Column

A 1 mg aliquot of the thrombinized his-tagged p14 (thmb-p14, see above) was covalently bound to 45 μl CNBr-activated Sepharose 4 FF beads (Amersham) under standard conditions as recommended by the company. Finally, the beads were washed three times in PBS.

A 1 ml aliquot of the cell lysate described above was bound to 20 μl of the affinity column by incubating them end over end for 1 hr at room temperature. The beads were then pelleted, washed and the bound proteins removed as described above for the Cobalt bound His-tagged p14 affinity column. In a separate experiment, the washes and elution of this column was varied. The first three sets of two washes each were identical, but the column was then washed 1× in 500 mM NaCl, 40 mM phosphate buffer pH 7.0, 1× in 700 mM NaCl, 40 mM phosphate buffer pH 7.0, and eluted in 600 μl 1500 mM NaCl, 40 mM phosphate buffer pH 7.0. The eluant was concentrated by precipitation in TCA, washed with acetone, dried and resuspended in SDS-PAGE sample buffer, run and analyzed as described above.

### In Vivo Treatment of Mice

a) Passive immunization As proof of principle, affinity purified Mab M-66 is used for passive immunization against T-25 lymphoma cells. Antibodies (in this case: affinity purified M-66 derived from ascites-10-20 mice inoculated with the M-66 hybridoma) are injected intraperitoneally (IP) every other day (1 mg/mouse/day, and 0.1 mg/mouse/day≈40 mg −4 mg/kg body weight) for 14 days (seven injections) starting one day after IP inoculation of the malignant cells (1×10⁶ cells/mouse) into syngeneic hosts. Mice (8-10 weeks old) are followed thereafter for clinical signs as well as histopathological manifestations of tumor growth and metastasis. No. of mice used: 10 (per group)×2 (control and experimental)×2(repetitions)=40 mice. In all in vivo studies, animals are taken care according to NIH guidelines for animal handling.

b) Active immunization Again, as proof of principle, p14 is used, to actively immunize Balb/C mice against MMTV-bearing tumors. Mice (two groups) are primed with purified recombinant p14 (100 μg and 25 μg/0.5 ml/mouse), and boosted after four weeks (again with 100 μg and 25 μg/mouse). Priming is carried out in Complete Freund's Adjuvant and boosting is with Incomplete Freund's Adjuvant. Sera of immunized mice are checked (3-4 weeks after the boost) for anti-p14 antibodies (using immunofluorescence and Western blotting). Mice, positive for p14 antibodies in their sera are challenged with highly tumorigenic T-25 cells at different inoculi (1×10⁶, 3×10⁵, 1×10⁵/0.2 ml/mouse, IP) and followed for tumor development. Impaired tumor growth in immunized mice, indicates that p14 can be used as a vaccine against MMTV bearing tumors.

c) Tumor targeting As a complementary approach to passive immunization, and as a proof of principle Mab M-66 is used for specific targeting of drugs (in their general meaning: Toxins, Radioactive isotopes, apoptosis inducing drugs, proapoptotic molecules, other bioactive molecules that can regulate the cell cycle etc.) to tumors that harbor MMTV. M-66 coupled to (37-72)Tat is used to target tumor cells in vivo (following IP inoculation).

### Preparation of Antibody-Tat Conjugate

(37-72)Tat Synthesis:

The (37-72) Tat fragment of HIV-HV1B1 Tat was synthesized and purified as described (Stein et al. FEBS letters, 458: 383-386, 1999; Adermann et al. FEBS letters, 453:173-177, 1998)

Synthesis of Mab M-66-(37-72)Tat Conjugate:

Solid sulfosuccinimidyl-6[3′-(2-pyridyldithio)propionamido]hexanoate (slcSPDP) is added to affinity purified M-66 in borate buffer (pH 8.5) to obtain a 3-6 fold molar excess over the protein concentration. Three hours are allowed for the acetylation. The reaction product (M-66-PDP) is purified by dialysis against PBS (pH 7.0). The PDP concentration is determined by incubating a sample with an excess of DTT and measuring the thiopyridon extinction at 343 nm. The 280 nm extinction, corrected for the contribution by PDP, is used to calculate the protein concentration. (37-72)Tat is added to obtain a 3-fold molar excess over the PDP concentration. At least 36 h are allowed for the thiol-disulfide exchange reaction. The resulting solution is purified by dialysis against PBS (pH 7.0). The protein concentration is determined from the 280 nm extinction.

Extinction coefficients used: E(IgG 10⁻⁵M, 1 cm, 280 nm)=2.1; E ((37-72) Tat, 10⁻⁵M, 1 cm, 280 nm)=0.012

### Humanization of Mouse Monoclonal Antibody M66

Total RNA is prepared from the M66 hybridoma cell line and reverse transcribed to cDNA. The heavy and light chains are being cloned by PCR using a battery of degenerate “forward” primers of the conserved signal sequence and “reverse” primers of the framework 4/constant region junction. The PCR products are being cloned into sequencing vectors and are being sequenced to confirm that the translated sequence corresponds to the antibody produced by the M66 hybridoma, The antibody is determined by N-terminal sequencing of the purified antibody.

These clones are used as the basis for cloning of the mouse variable regions of the M66 monoclonal antibody into expression cassettes of human constant regions essentially as described by Meyuhas et al “HIV-1 neutralization by chimeric CD4-CG10 polypeptides fused to human IgG1” Mol. Immunol. In press (2005).

Intact humanized M66. The mouse heavy variable region is inserted into a cassette including human constant regions CH1, CH2 and CH3. The mouse light variable region is inserted into a cassette including Cλ2. The resulting plasmids are then co-transfected into SP2/0 myeloma cells.

M66(scFv)-hIgG1. The heavy variable region is connected to the light variable chain using the 218 linker (GSTSGSGKPGSGEGSTKG), (Whitlow et al “An improved linker for single chain Fv with reduced aggregation and enhanced proteolytic stability” Protein Eng. 6 989-995 (1993)) and is inserted into a cassette including human constant regions CH2 and CH3. The resulting plasmid is then transfected into SP2/0 cells and the two mouse variable regions are expressed on the same molecule.

In both instances (Intact humanized M66 and M66(scFv)-hIgG1) stable transfectants are selected and the secreted antibodies are purified from the supernatant using Protein A-sepharose.

1-39. (canceled)

40. A method for diagnosing breast cancer in a subject comprising determining levels of expression of p14 peptide in one or more samples from said subject, a high level of expression signifying a high probability for breast cancer in said subject.

41. The method of claim 40, comprising assaying for the level of p14 peptide in a sample obtained from the subject, said method comprises:
(a) contacting said sample with anti-p14 antibodies;
(b) determining binding of anti-p14 antibodies to p14 peptide.

42. The method of claim 41, wherein said sample is a tissue or body fluid sample excised or withdrawn from a suspicious area in the breast of the subject.

43. The method of claim 42, wherein said sample is selected from fresh biopsy section, cryo-section or paraffin embedded section.

44. The method of claim 40, wherein said sample is a blood sample.

45. The method of claim 40, comprising assaying for the level of anti-p14 antibodies in a sample obtained from the subject, said method comprises:
(a) contacting said sample with p14 peptide;
(b) determining binding of p14 peptide to anti-p14 antibodies.

46. The method of claim 45, wherein said p14 peptide is His-tag p14 peptide comprising the sequence depicted in SEQ ID NO:2.

47. A method for screening samples into such which signify that subjects from which they were obtained have a relatively high possibility of having or being susceptible of developing breast cancer and such which signify that subjects from which they were obtained have a relatively lower probability of having or being susceptible of developing breast cancer, the method comprising contacting the samples with anti-p14 antibodies and determining binding of anti-p14 antibodies and p14 peptide in said sample, a high degree of binding signifying a corresponding higher probability of having or being susceptible of developing breast cancer.

48. The method of claim 47, wherein said sample is a tissue or fluid sample excised or withdrawn from a suspicious area in the breast of the subject.

49. The method of claim 48, wherein said sample is selected from fresh biopsy section, cryo-section or paraffin embedded section.

50. The method of claim 49, wherein said sample is a blood sample.

51. A method for screening samples into such which signify that subjects from which they were obtained have a relatively high possibility of having or being susceptible of developing breast cancer and such which signify that subjects from which they were obtained have a relatively lower probability of having or being susceptible of developing breast cancer, the method comprising contacting the samples with p14 peptide and determining binding of p14 peptide with anti-p14 antibodies, a high degree of binding signifying a corresponding higher probability of having or being susceptible of developing breast cancer.

52. The method of claim 51, wherein said sample is a blood sample.

53. The method of claim 52, wherein said p14 peptide is His-tag p14 peptide comprising the sequence depicted in SEQ ID NO:2.

54. A method for the treatment of breast cancer comprising administering to a subject in need of anti-breast cancer treatment an amount of anti-p14 antibodies, the amount being sufficient to achieve an anti cancer effect in said subject.

55. The method of claim 54, wherein said anti-p14 antibodies are humanized antibodies.

56. The method of claim 54, wherein said anti-p14 antibodies are bound to a protein transducing element.

57. The method of claim 56, wherein said protein transducing element is the (37-72) Tat fragment of HIV-HV1B1 Tat.

58. The method of claim 54, wherein said anti-p14 antibodies are bound to a cytotoxic agent.

59. A method for the treatment of breast cancer comprising administering to a subject in need an amount of p14 peptide, the amount being effective to elicit production of anti-p14 antibodies in said subject.

60. A pharmaceutical composition for the treatment of breast cancer comprising as active ingredient an amount of anti-p14 antibodies, the amount being sufficient to achieve a therapeutic effect in said subject.

61. The pharmaceutical composition of claim 60, wherein said anti-p14 antibodies are humanized antibodies.

62. The pharmaceutical composition of claim 61, wherein said anti-p14 antibodies are bound to a protein transducing element.

63. The pharmaceutical composition of claim 62, wherein said protein transducing element is the (37-72) Tat fragment of HIV-HV1B1 Tat.

64. The pharmaceutical composition of claim 60, wherein said anti-p14 antibodies are bound to a cytotoxic agent.

65. A vaccine comprising as active ingredient an amount of p14 peptide or an immunogenic fragment thereof, the amount being sufficient to elicit in a subject production of anti-p14 antibodies.
